(19) United States
(12) Patent Application Publication
(10) Pub. No.: US 2021/0315799 A1
(43) Pub. Date: Oct. 14, 2021

(54) Composition for Promoting Differentiation or Proliferation of Human Adipose-Derived Stem Cell

(71) Applicant: LG Household & Health Care Ltd., Seoul (KR)

(72) Inventors: Seonju Lee, Seoul (KR); Mina Kim, Seoul (KR); Mi Sun Kim, Seoul (KR)

(73) Assignee: LG Household & Health Care Ltd., Seoul (KR)

(21) Appl. No.: 17/270,697

(22) PCT Filed: Aug. 23, 2019
(86) PCT No.: PCT/KR2019/010815

§ 371 Date: Feb. 23, 2021

(30) Foreign Application Priority Data
Aug. 23, 2018 (KR) 10-2018-0098717
Sep. 12, 2018 (KR) 10-2018-0109108
Mar. 15, 2019 (KR) 10-2019-0029998

(51) Int. Cl. A61K 8/9789; C12N 5/077; A23L 33/105; A61K 36/57; A61Q 19/00; A61Q 19/08

(52) U.S. Cl. CPC A61K 8/9789; C12N 5/0653; A23L 33/105; A61K 2800/805; A61Q 19/001; A61Q 19/08; A61K 36/57

(57) ABSTRACT

Provided are a cosmetic composition, a food composition, a pharmaceutical composition, and an over-the-counter (OTC) composition for skin elasticity enhancement and skin wrinkle reduction, a composition for promoting differentiation or proliferation of human adipose-derived stem cells, and a method of enhancing skin elasticity including administering the compositions to an individual other than a human, each composition including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

### TECHNICAL FIELD

The present invention relates to a cosmetic composition, a food composition, a pharmaceutical composition, and an over-the-counter (OTC) composition for skin elasticity enhancement and skin wrinkle reduction, a composition for promoting differentiation or proliferation of human adipose-derived stem cells, and a method of enhancing skin elasticity including administering the compositions to an individual other than a human, each composition including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

### BACKGROUND ART

Adipose tissue serves as a support framework for skin together with muscles. Although the number of adipocytes accounts for 30% to 40%, adipocytes are structurally important cells in the skin occupying 90% by volume. It is known that subcutaneous adipose tissue decreases with aging of an individual, and thus skin elasticity decreases and wrinkles increase. In addition, when a body fat level is lowered due to rapid weight loss, a decrease in skin elasticity of a facial area and an increase in wrinkles are observed in many cases. Therefore, skin elasticity may be enhanced and wrinkles reduced by increasing volume inside the skin by adjusting the number of adipose tissues, which have an important role in maintaining the structure inside skin. However, in the case of fat grafting procedures currently performed to enhance skin elasticity, problems of a very low in vivo engraftment rate of grafted stem cells, a likelihood of reoperation after some period of time, and clumping of grafted fat may occur. Therefore, research is necessary into substances capable of strengthening the structure of the subcutaneous layer without a surgical procedure.

However, in the case of conventional technology of directly injecting adipose-derived stem cells, problems of safety may occur. In the case of technology of imitating components secreted during a cell cultivation process and supplying the components to skin, it is difficult to actually replicate the components. In addition, as skin ages, activity of human adipose-derived stem cells decreases and time for proliferation increases two-fold or more (Gerontolog 2011; 57:66-75), and thus adipose stem cells present in aging skin have greatly reduced reactivity compared to other application technologies. Thus, research is required into substances activating subcutaneous adipose tissue by promoting proliferation of adipose stem cells without causing skin irritation.

In addition, the skin of lips, one of the most frequently moving body parts due to repeated contraction and expansion, is always exposed to extreme environments and easily affected by environmental stimulation such as ultraviolet rays, cold, and dryness. Also, with a structure different from that of normal skin, the skin of the lips has low self-defense ability against environmental stimulation, resulting in decreases in volume and increases in wrinkles as the skin ages, thereby losing an attractive appearance thereof. Therefore, there is a need to develop compositions having excellent effects on volume and wrinkle care specialized for lips as in the normal skin. However, only technology using the same wrinkle-reducing components developed for facial skin and technology temporarily increasing the volume of the lips have been used. The increase in volume via the latter is limited since it is caused by a stimulus reaction (Korean Patent Laid-open Publication No. 10-2012-0140450 A).

In recent years, adipose-derived stem cells that are present in subcutaneous adipose tissue and have the ability to differentiate into adipose cells have drawn attention in studies related to adipose cells or fat grafting technology. This is because adipokines secreted by adipose-derived stem cells are known to not only increase collagen synthesis in the dermal layer but also affect skin regeneration. Since substances known to differentiate adipocyte progenitor cells into adipose cells have been verified based on results of experiments performed on mice, efficiency thereof is low when applied to human adipose-derived stem cells.

### DISCLOSURE

### Technical Problem

As a result of intensive efforts to develop substances capable of effectively promoting differentiation of adipose-derived stem cells into adipose cells or proliferation of adipose-derived stem cells, the present inventors have developed formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, and Aloe vera extract, as substances capable of promoting differentiation of adipose-derived stem cells into adipose cells and confirmed that these substances may be used for the purpose of skin elasticity enhancement and skin wrinkle reduction, thereby completing the present invention.

### Technical Solution

An object of the present invention is to provide a cosmetic composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

Another object of the present invention is to provide a food composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, Aloe vera extract, fractions of the Magnoliae cortex extract, and fractions of the Aloe vera extract as an active ingredient.

Another object of the present invention is to provide a pharmaceutical composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

Another object of the present invention is to provide an over-the-counter composition (OTC) composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

Another object of the present invention is to provide a composition for promoting differentiation of human adipose-derived stem cells including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

Another object of the present invention is to provide a method of enhancing skin elasticity, the method including administering the compositions to an individual other than a human.

### Advantageous Effects

The compositions of the present invention may be used for anti-aging purposes by strengthening the subcutaneous layer structure, enhancing skin elasticity, and reducing skin wrinkles by promoting differentiation of human adipose-derived stem cells into adipose cells or proliferation of human adipose-derived stem cells.

### BRIEF DESCRIPTION OF DRAWINGS

FIG. 1 shows results of treating human adipose-derived stem cells with formononetin, acetyl mandelic acid, and Magnoliae cortex extract, and then staining fats and lipids of differentiated adipose cells using Oil Red 0.

FIG. 2 quantitatively shows relative differentiation rates of human adipose-derived stem cells treated with formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, and Aloe vera extract.

FIG. 3 shows comparison results of the numbers of people having reduced wrinkles around eyes and in lips when treated with Magnoliae cortex extract.

FIG. 4A shows duration of effects on reducing wrinkles in lips of Subject 1 treated with Magnoliae cortex extract until the 12th week, and FIG. 4B shows duration of effects on reducing wrinkles in lips of Subject 2 until the 12th week.

FIG. 5 shows results of comparison between eye and lip wrinkle reduction rates of Magnoliae cortex extract and asiaticoside, which is a conventional wrinkle-reducing substance.

FIG. 6 shows cell activity rates of human adipose-derived stem cells treated with raspberry ketone, gallic acid, caffeic acid, carnosic acid, terpineol, sodium mannose phosphate, tropolone, glycyrrhetinic acid, lanosterol, sodium riboflavin phosphate, menadione, thioctic acid, lactoflavin, xanthophyll, and extracts of onion, rice bran, bamboo, carrot, and Hibiscus.

### BEST MODE

The present invention will be described in detail. Meanwhile, each description and embodiment disclosed in the present invention may be applied herein to different descriptions and embodiments. In other words, all combinations of various components disclosed in the present invention are included within the scope of the present invention. Furthermore, the scope of the present invention should not be limited by the descriptions provided below.

An aspect of the present invention provides a cosmetic composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient. The composition may include 1, 2, 3, 4, 5, 6, 7, 8, 9, 10, 11, 12, 13, 14, 15, or 16 substances among the above-described substances as active ingredients.

In addition, according to an embodiment of the present invention, provided is a cosmetic composition for skin elasticity enhancement and skin wrinkle reduction including a cosmetically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll as an active ingredient.

In the present invention, the “cosmetically acceptable salt” is in the form of a salt in which formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll is combined with another substance, and refers to a substance capable of exhibiting cosmetically similar activity to that of the compounds, without being limited thereto.

As a result of intensive efforts to develop substances capable of activating and restoring aged subcutaneous layers, the present inventors have confirmed that formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, and Aloe vera extract promote differentiation of human adipose-derived stem cells into adipose cells and proliferation of human adipose-derived stem cells. Based thereon, it can be seen that compositions including the substances may be used as cosmetic compositions for skin elasticity enhancement and skin wrinkle reduction. The effect of these substances on skin elasticity enhancement and skin wrinkle reduction has not been known at all, and it is very significant that this has been first developed by the present inventors.

As used herein, the term “formononetin” refers to a substance having a molecular formula of C₁₆H₁₂O₄ and a molecular weight of 284.26, and may be represented by Formula 1 below.

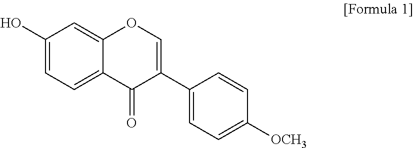 [SMILES: COc1ccc(-c2coc3cc(O)ccc3c2=O)cc1]

The formononetin of the present invention may be isolated from plants such as licorice, but methods of obtaining formononetin are not particularly limited, and formononetin chemically synthesized using any known method or commercially purchased may be used.

As used herein, the term “emodin” refers to a substance having a molecular formula of C₁₅H₁₀O₅ and a molecular weight of 270.24, and may be represented by Formula 2 below.

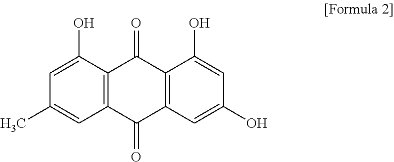 [SMILES: Cc1cc(O)c2c(c1)C(=O)c1cc(O)cc(O)c1C2=O]

Methods of obtaining the emodin of the present invention are not particularly limited, and emodin chemically synthesized using any known method or commercially purchased may be used.

As used herein, the term “artepillin C” refers to a substance having a molecular formula of C₁₉H₂₄O₃ and a molecular weight of 300.40, and may be represented by Formula 3 below.

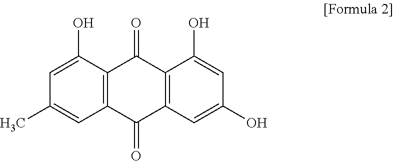 [SMILES: Cc1cc(O)c2c(c1)C(=O)c1cc(O)cc(O)c1C2=O]

Methods of obtaining the artepillin C of the present invention are not particularly limited, and artepillin C chemically synthesized using any known method or commercially purchased may be used.

As used herein, the term “carnosic acid” refers to a substance having a molecular formula of C₂₀H₂₈O₄ and a molecular weight of 332.44, and may be represented by Formula 4 below.

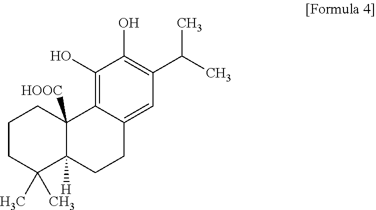 [SMILES: CC(C)c1cc2c(c(O)c1O)[C@@]1(C(=O)O)CCCC(C)(C)[C@@H]1CC2]

Methods of obtaining the carnosic acid of the present invention are not particularly limited, and carnosic acid chemically synthesized using any known method or commercially purchased may be used.

As used herein, the term “palmitamide”, as a normal chain fatty acid amide including 16 carbon atoms, has a molecular formula of C₁₆H₃₃NO and a molecular weight of 255, and may be represented by Formula 5 below.

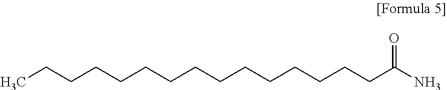 [SMILES: CCCCCCCCCCCCCCCC(N)=O]

Methods of obtaining the palmitamide of the present invention are not particularly limited, and palmitamide chemically synthesized using any known method or commercially purchased may be used.

As used herein, the term “sodium guaiazulene sulfonate” refers to a substance having a molecular formula of C₁₅H₁₇NaO₃S and a molecular weight of 300.35, and may be represented by Formula 6 below.

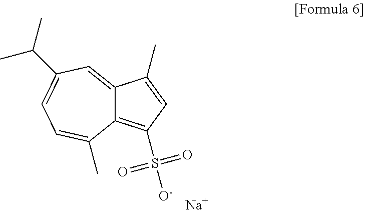 [SMILES: Cc1cc(S(=O)(=O)[O-])c2c(C)ccc(C(C)C)cc1-2 ; [Na+]]

Methods of obtaining the sodium guaiazulene sulfonate of the present invention are not particularly limited, and sodium guaiazulene sulfonate chemically synthesized using any known method or commercially purchased may be used.

As used herein, the term “caffeic acid” refers to a substance having a molecular formula of C₉H₈O₄ and a molecular weight of 180.157, and may be represented by Formula 7 below.

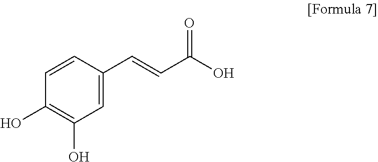 [SMILES: O=C(O)/C=C/c1ccc(O)c(O)c1]

Methods of obtaining the caffeic acid of the present invention are not particularly limited, and caffeic acid chemically synthesized using any known method or commercially purchased may be used.

As used herein, the term “acetyl mandelic acid” may refer to, specifically, “O-acetyl mandelic acid”, which has a molecular formula of C₁₀H₁₀O₄ and a molecular weight of 194.186, and may be represented by Formula 8 below.

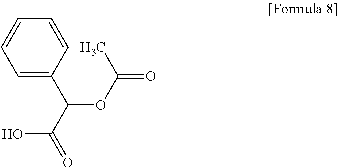

Methods of obtaining the acetyl mandelic acid of the present invention are not particularly limited, and acetyl mandelic acid chemically synthesized using any known method or commercially purchased may be used.

As used herein, the term “laccaic acid” refers to a natural anthraquinone, as a dark red crystal prepared by coagulating secretion of Laccifer Iacca Kerr, and is known to be used as a dye. The laccaic acid may be laccaic acid A, B, C, D, E, or F, but is not limited thereto. In an embodiment of the present invention, product 50506 (Natural red 25) of Sigma-Aldrich was used as laccaic acid.

Methods of obtaining the laccaic acid of the present invention are not particularly limited, and laccaic acid chemically synthesized using any known method or commercially purchased may be used.

As used herein, the term “ubiquinone”, one of fat-soluble quinones, refers to a component of the mitochondrial electron transport system. Specifically, the ubiquinone may be ubiquinone 10, but is not limited thereto. In this case, the ubiquinone may have a molecular formula of C₅₉H₉₀O₄ and a molecular weight of 863.37, and may be represented by Formula 9 below.

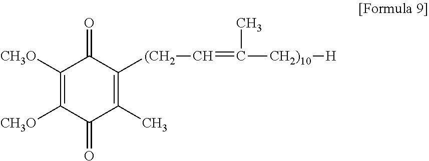

Methods of obtaining the ubiquinone are not particularly limited, and ubiquinone chemically synthesized using any known method or commercially purchased may be used.

As used herein, the term “hydroxycinnamic acid” refers to a substance having a molecular formula of C₉H₈O₃ and a molecular weight of 164.158, and may include all isomers (o-, m-, and p-hydroxycinnamic acid) according to the position of the —OH group directly substituted at a benzene ring and isomers (cis-type coumarinic acid and trans-type coumaric acid) according to a double bond. Specifically, hydroxycinnamic acid may be m-coumaric acid, without being limited thereto, and may be represented by Formula 10 below.

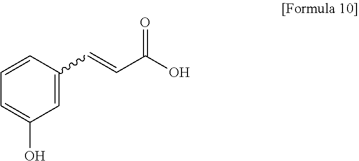 [SMILES: O=C(O)C=Cc1cccc(O)c1]

Methods of obtaining the hydroxycinnamic acid of the present invention are not particularly limited, and hydroxycinnamic acid chemically synthesized using any known method or commercially purchased may be used.

As used herein, the term “xanthophyll” refers to a carotenoid-based oxycarotenoid pigment with yellow color present in green parts of plants, such as leaves, flowers, and fruits, and which has a molecular formula of C₄₀H₅₆O₂ and a molecular weight of 569. The xanthophyll may be lutein and may be represented by Formula 11 below.

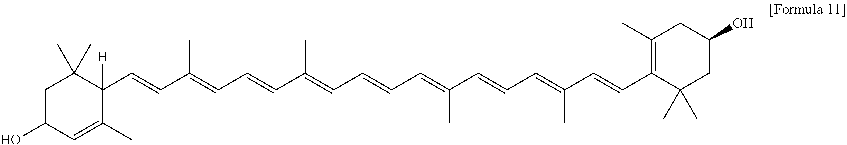 [SMILES: CC1=CC(O)CC(C)(C)C1/C=C/C(C)=C/C=C/C(C)=C/C=C/C=C(C)/C=C/C=C(C)/C=C/C1=C(C)C[C@@H](O)CC1(C)C]

Methods of obtaining the xanthophyll or lutein of the present invention are not particularly limited, and xanthophyll or lutein chemically synthesized using any known method or commercially purchased may be used.

As used herein, the term “raspberry ketone” refers to a substance having a molecular formula of C₁₀H₁₂O₂ and a molecular weight of 164.20, and may be represented by Formula 1 below.

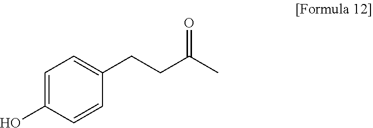

Methods of obtaining the raspberry ketone of the present invention are not particularly limited, and raspberry ketone chemically synthesized using any known method or commercially purchased may be used.

As used herein, the term “gallic acid” refers to a substance having a molecular formula of C₇H₆O₅ and a molecular weight of 170.12, and may be represented by Formula 13 below.

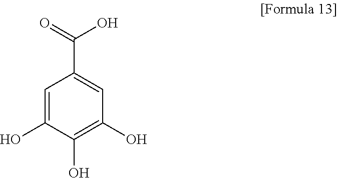 [SMILES: O=C(O)c1cc(O)c(O)c(O)c1]

Methods of obtaining the gallic acid of the present invention are not particularly limited, and gallic acid chemically synthesized using any known method or commercially purchased may be used.

As used herein, the term “terpineol” refers to a substance having a molecular formula of C₁₀H₁₈O and a molecular weight of 154, and may be represented by Formula 14 below.

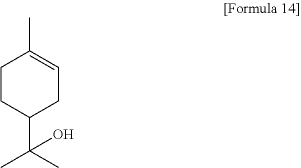

Methods of obtaining the terpineol of the present invention are not particularly limited, and terpineol chemically synthesized using any known method or commercially purchased may be used.

As used herein, the term “sodium mannose phosphate” refers to a substance having a molecular formula of C₆H₁₂O₉PNa and a molecular weight of 282.12. Specifically, the sodium mannose phosphate may be sodium mannose 6-phosphate or D-mannose-6-phosphate sodium salt, without being limited thereto, and may be represented by Formula 15 below.

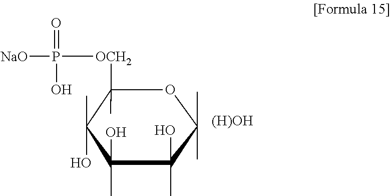

Methods of obtaining the sodium mannose phosphate of the present invention are not particularly limited, and sodium mannose phosphate chemically synthesized using any known method or commercially purchased may be used.

As used herein, the term “tropolone” refers to a substance having a molecular formula of C₇H₆O₂ and a molecular weight of 122, and may be represented by Formula 16 below.

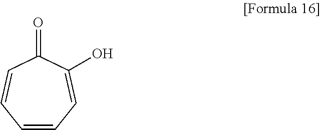

Methods of obtaining the tropolone of the present invention are not particularly limited, and tropolone chemically synthesized using any known method or commercially purchased may be used.

As used herein, the term “glycyrrhetinic acid” refers to a substance having a molecular formula of C₃₀H₄₆O₄ and a molecular weight of 471, and may be represented by Formula 17 below.

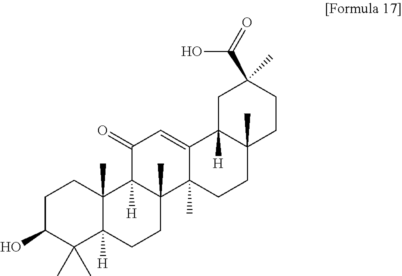

Methods of obtaining the glycyrrhetinic acid of the present invention are not particularly limited, and glycyrrhetinic acid chemically synthesized using any known method or commercially purchased may be used.

As used herein, the term “lanosterol” refers to a substance having a molecular formula of C₃₀H₅₀O and a molecular weight of 427, and may be represented by Formula 18 below.

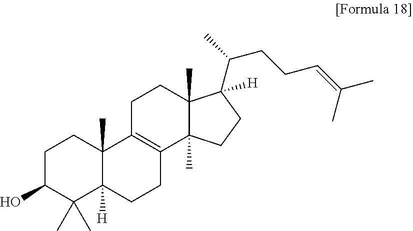 [SMILES: CC(C)=CCC[C@@H](C)[C@H]1CC[C@@]2(C)C3=C(CC[C@]12C)[C@@]1(C)CC[C@H](O)C(C)(C)[C@@H]1CC3]

Methods of obtaining the lanosterol of the present invention are not particularly limited, and lanosterol chemically synthesized using any known method or commercially purchased may be used.

As used herein, the term “sodium riboflavin phosphate” refers to a substance having a molecular formula of C₁₇H₂₀N₄NaO₉P and a molecular weight of 478.33, and may be represented by Formula 19 below.

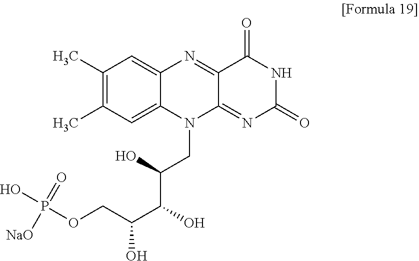

Methods of obtaining the sodium riboflavin phosphate of the present invention are not particularly limited, and sodium riboflavin phosphate chemically synthesized using any known method or commercially purchased may be used.

As used herein, the term “menadione”, as a kind of vitamin K, refers to a yellow crystal having a molecular formula of C₁₁H₁₈O₂ and a molecular weight of 172.18, and may be represented by Formula 20 below.

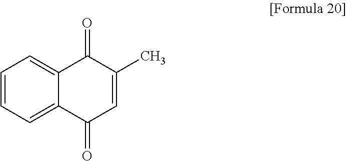

Methods of obtaining the menadione of the present invention are not particularly limited, and menadione chemically synthesized using any known method or commercially purchased may be used.

As used herein, the term “thioctic acid” is also referred to as lipoic acid, has a molecular formula of C₈H₁₄O₂S₂ and a molecular weight of 206, and may be represented by Formula 21 below.

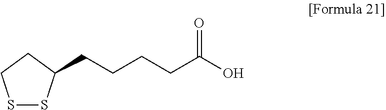 [SMILES: O=C(O)CCCC[C@@H]1CCSS1]

Methods of obtaining the thioctic acid or lipoic acid of the present invention are not particularly limited, and thioctic acid or lipoic acid chemically synthesized using any known method or commercially purchased may be used.

As used herein, the term “lactoflavin” refers to riboflavin (vitamin B₂) isolated from milk. The lactoflavin of the present invention has a molecular formula of C₁₇H₂₀N₄O₆ and a molecular weight of 376.36, and may be represented by Formula 22 below.

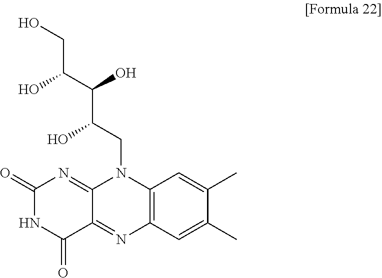 [SMILES: Cc1cc2nc3c(=O)[nH]c(=O)nc-3n(C[C@H](O)[C@H](O)[C@H](O)CO)c2cc1C]

Methods of obtaining the lactoflavin of the present invention are not particularly limited, and lactoflavin chemically synthesized using any known method or commercially purchased may be used.

The formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll may be present not only in a solvated form but also in an unsolvated form. Also, the compound may be present in a crystalline or amorphous form, and all of the physical forms are included in the scope of the present invention.

In addition, it is obvious that not only the compounds described above but also cosmetically, sitologically, and pharmaceutically acceptable salts thereof are included in the scope of the compounds.

As used herein, the term “onion (Allium cepa)” refers to a biennial plant, a monocotyledonous plant in the Liliaceae family belonging to the order of Veneroida, and is known to have anti-inflammatory, blood sugar level-controlling, and blood pressure-lowering effects due to quercetin, chromium, allicin, and the like contained therein as main ingredients. Any commercially available onions may be purchased, or onions harvested in nature or grown may be used without limitation, and roots, stems, and leaves thereof may be used without limitation.

As used herein, the term “rice bran” refers to a pulverized mixture of pericarp, seed coat, and aleurone layer produced when brown rice is polished to produce polished rice, and is known to have cholesterol level-lowering, anti-oxidant, and anti-cancer effects due to vitamins B₁ and B₂, minerals such as niacin, oryzanol, tocopherol, phytic acid, and the like contained therein.

As used herein, the term “bamboo (Bambusoideae)” refers to an evergreen perennial plant, a monocotyledonous plant in the subfamily Bambusoideae of the grass family Poaceae, and is known to have anti-oxidant, sterilizing, and anti-cancer effects in oriental medicine. Any commercially available bamboo may be purchased, or bamboo harvested in nature or grown may be used without limitation, and roots, stems, and leaves thereof may be used without limitation.

As used herein, the term “carrot (Daucus carota var. sativa)” refers to a biennial plant, a dicotyledonous plant in the Apiaceae family belonging to the order of Apiales, and is known to have anti-oxidant, anti-aging, and immunity-strengthening effects due to beta-carotene, lycopene, and the like contained therein as primary components. Any commercially available carrots may be purchased, or carrots harvested in nature or grown may be used without limitation, and roots, stems, and leaves thereof may be used without limitation.

As used herein, the term “Hibiscus (Rose of China)” refers to a dicotyledonous plant in the Mallow family belonging to the order of Malvales and is known to have effects on obesity prevention due to abundant Hibiscus acid, hydroxycitric acid, and the like. Any commercially available Hibiscus may be purchased, or Hibiscus harvested in nature or grown may be used without limitation, and roots, stems, and leaves thereof may be used without limitation.

As used herein, the term “Magnoliae cortex” refers to dried stems or root barks of Machilus tree in the Magnolia family, includes lignan and sesame component as main active ingredients. Magnoliae cortex has been known to have anti-inflammatory, analgesic, smooth muscle-regulating, and anti-ulcer effects. Magnoliae cortex commercially purchased or harvested in nature or grown may be used without limitation.

As used herein, the term “Aloe vera” is a perennial plant belonging to the genus Aloe in the Asphodelaceae family. Aloe vera includes aloin, Aloe emodin, aloetin, alomicin, and the like as main active ingredients and has been known to have antibacterial, antifungal, and antitumor activity. Aloe vera commercially purchased or harvested in nature or grown may be used without limitation.

As used herein, the term “extract” refers to a resultant such as liquid components obtained by immersing a target substance in various solvents and performing extraction at room temperature, a low temperature, or a high temperature for a predetermined period of time, or solids obtained by removing the solvents from the liquid components. Also, in addition to the resultant, diluents, concentrations, crude purified products, or purified products may also be inclusively understood as the extract.

In the present invention, the extract may be extract of onion, rice bran, bamboo, carrot, Hibiscus, Magnoliae cortex, or Aloe vera. The extract may be obtained from various organs of natural, hybrid, and variant plants, for example, roots, aboveground parts, stems, leaves, flowers, trunks of fruits, and skins of fruits as well as plant tissues. The extract may be obtained using water or various organic solvents. In this case, the organic solvents are not particularly limited as long as the extract is obtainable using the organic solvents, but may be, specifically, water, polar solvents, or non-polar solvents, more specifically, water, a C₁-C₆ lower alcohol (such as methanol, ethanol, propanol, or butanol), a polyhydric alcohol such as glycerin and butylene glycol, a hydrocarbon-based solvent such as methyl acetate and ethyl acetate, or any mixed solvent thereof, and even more specifically, water, a lower alcohol, 1,3-butylene glycol, or any mixed solvent thereof.

In addition, methods of obtaining the extract are not particularly limited as long as the extract of the onion, rice bran, bamboo, carrot, Hibiscus, Magnoliae cortex, or Aloe vera is obtainable using the methods. Specifically, after immersing roots, stems, leaves, fruits, or flowers of onion, rice bran, bamboo, carrot, Hibiscus, Magnoliae cortex, or Aloe vera, dried products or processed products thereof, or the like in the above-described solvents, extraction methods such as enfleurage performed at room temperature, maceration, ultrasonic extraction performed by applying ultrasonic waves, and reflux extraction performed using a reflux cooler may be used.

As used herein, the term “fraction” refers to a resultant obtained by performing fractionation to isolate a particular component or a particular component group from a mixture of various components. In the present invention, the fractions may be fractions of the onion extract, rice bran extract, bamboo extract, carrot extract, Hibiscus extract, Magnoliae cortex extract, or Aloe vera extract.

In the present invention, a fractionation method to obtain the fraction is not particularly limited as long as the fraction has the effects on skin elasticity enhancement and skin wrinkle reduction, and any methods commonly available in the art may also be used. For example, a solvent fractionation method performed by treating with various solvents, an ultrafiltration fractionation method performed by passing through an ultrafiltration membrane having a specific molecular weight cut-off value, a chromatographic fractionation method performed by using various chromatographic systems (manufactured for separation based on size, charge, hydrophobicity, or affinity), and any combination thereof may be used.

In the present invention, the type of a fractionation solvent used to obtain the fraction is not particularly limited, and any solvent well known in the art may be used. Non-limiting examples of the fractionation solvent may include: a polar solvent such as water, distilled water, and alcohol; and a non-polar solvent such as hexane, ethyl acetate, chloroform, and dichloromethane. These solvents may be used alone or in a combination of at least two thereof. When alcohol is used as the fractionation solvent, a C₁-C₄ alcohol may be used.

The cosmetic composition of the present invention may include at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, fractions of the Magnoliae cortex extract, and fractions of the Aloe vera extract in an amount of 0.0001 wt % to 10 wt % (w/w), specifically, 0.0005 wt % to 10 wt % (w/w), 0.001 wt % to 5 wt % (w/w), or 0.0001 wt % to 2 wt % (w/w) based on a total weight of the composition, without being limited thereto. When the amount of the substance is less than 0.0001 wt %, the effects of the substance may be negligible, and when the amount of the substance is equal to or greater than 2 wt %, cytotoxicity may be caused.

In the present invention, the cosmetic composition may promote differentiation of human adipose-derived stem cells into adipose cells or proliferation of human adipose-derived stem cells. That is, the cosmetic composition may enhance skin elasticity and reduce skin wrinkles by promoting differentiation of human adipose-derived stem cells into adipose cells or proliferation of human adipose-derived stem cells.

As used herein, the term “human adipose-derived stem cell” refers to a stem cell isolated from human adipose tissue as a multipotent cell that may differentiate into multiple cells of the human body. The adipose-derived stem cells are present in the subcutaneous adipose tissue and are capable of differentiating into adipose cells. Adipokines secreted by the adipose-derived stem cells are known to not only promote synthesis of collagen in the dermal layer but also affect skin regeneration.

As used herein, the term “adipose cell” refers to a cell that stores energy as fat in adipose tissue of an individual, specifically white adipocytes present in the subcutaneous layer, but is not limited thereto.

In an embodiment of the present invention, as a result of treating human adipose-derived stem cells with formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, and Aloe vera extract, it was confirmed that these substances effectively promote differentiation of human adipose-derived stem cells into adipose cells. Therefore, the compositions of the present invention including these substances may be used for anti-aging purposes by enhancing skin elasticity and reducing skin wrinkles.

In an embodiment of the present invention, as a result of treating human adipose-derived stem cells with raspberry ketone, gallic acid, caffeic acid, carnosic acid, terpineol, sodium mannose phosphate, tropolone, glycyrrhetinic acid, lanosterol, sodium riboflavin phosphate, menadione, thioctic acid, lactoflavin, xanthophyll, and extracts of onion, rice bran, bamboo, carrot, and Hibiscus, it was confirmed that cell activity rates of the human adipose-derived stem cells significantly increased due to the treatment compared to a control, resulting in promoting proliferation thereof. Thus, the composition of the present invention including these substances may be used for the purpose of anti-aging by enhancing skin elasticity and reducing skin wrinkles.

As used herein, the term “enhancement of skin elasticity” refers to improvement in elasticity of skin by strengthening the structure of the subcutaneous layer of an individual using the composition of the present invention. The position of the skin on an individual is not particularly limited as long as skin elasticity may be enhanced or skin wrinkles are reduced by applying the composition of the present invention thereto.

As used herein, the term “skin wrinkle reduction” refers to alleviating symptoms of skin wrinkles of an individual using the composition of the present invention.

Another aspect of the present invention provides a cosmetic composition for elasticity enhancement and wrinkle reduction of lip skin including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, Aloe vera extract, fractions of the Magnoliae cortex extract, and fractions of the Aloe vera extract as an active ingredient.

According to an embodiment of the present invention, the cosmetic composition for elasticity enhancement and wrinkle reduction of lip skin may include at least one substance selected from Magnoliae cortex extract and fractions of the Magnoliae cortex extract.

Unlike other parts of skin, lips have no sebaceous glands and have a very thin stratum corneum. Most of the components currently used for volume increase and wrinkle reduction of lips are applied as components for enhancing the dermal layer. Thus, technology for improving the subcutaneous layer, which occupies the greatest volume in the skin, has not been widely used. In addition, considering that the stratum corneum of the lips is very thin, and thus penetration and delivery of the active ingredient are easier than in other parts of the skin, technology of increasing volume of the subcutaneous layer may be expected to be more effective in terms of volume increase and wrinkle reduction. The composition of the present invention is characterized by having excellent effects on lip skin. In an embodiment of the present invention, it was confirmed that the Magnoliae cortex extract has excellent effects on reducing wrinkles around eyes and in lips and higher wrinkle reduction speed and wrinkle reduction rate in lip wrinkles than in wrinkles around the eyes.

The composition for lip skin may be prepared in a formulation selected from a lipstick, a lip gloss, a lip tint, or a lip balm, without being limited thereto.

In the present invention, the cosmetic composition may be prepared into a formulation selected from the group consisting of a solution, ointment for external skin use, cream, foam, nourishing lotion, softening lotion, pack, skin softener, milky lotion, makeup base, essence, soap, liquid cleanser, bath bomb, sun screen cream, sun oil, suspension, emulsion, paste, gel, lotion, powder, surfactant-containing cleansing agent, oil, powder foundation, emulsion foundation, wax foundation, patch, and spray, without being limited thereto.

In addition, the cosmetic composition of the present invention may further include at least one cosmetically acceptable carrier appropriately mixed with general skin cosmetic formulations, such as oil, water, a surfactant, a moisturizing agent, a lower alcohol, a thickener, a chelating agent, a pigment, a preservative, or a fragrance, without being limited thereto.

The cosmetically acceptable carrier included in the cosmetic composition of the present invention may vary according to the formulation.

When the formulation of the present invention is an ointment, paste, cream, or gel, the carrier may be animal oil, vegetable oil, wax, paraffin, starch, tragacanth, a cellulose derivative, polyethylene glycol, silicone, bentonite, silica, talc, zinc oxide, or any mixture thereof.

When the formulation of the present invention is a powder or spray, the carrier may be lactose, talc, silica, aluminum hydroxide, calcium silicate, polyamide powder, or any mixture thereof. In particular, in the form of a spray, the cosmetic composition may further include a propellant such as a chlorofluorohydrocarbon, propane/butane, or dimethyl ether.

When the formulation of the present invention is a solution or emulsion, the carrier may be a solvent, a solubilizer, or an emulsifier, e.g., water, ethanol, isopropanol, ethyl carbonate, ethyl acetate, benzyl alcohol, benzyl benzoate, propylene glycol, and 1,3-butyl glycol oil, particularly cotton seed oil, peanut oil, corn seed oil, olive oil, castor oil, sesame oil, glycerol, an aliphatic ester, polyethylene glycol, or a fatty acid ester of sorbitan.

When the formulation of the present invention is a suspension, the carrier may be a liquid diluent such as water, ethanol, and propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth.

When the formulation of the present invention is a soap, the carrier may be an alkali metal salt of a fatty acid, a hemiester salt of a fatty acid, a fatty acid protein hydrolysate, isethionate, a lanolin derivative, an aliphatic alcohol, vegetable oil, glycerol, or sugar.

The cosmetic composition of the present invention may further include an adjuvant commonly used in the art, e.g., a hydrophilic or lipophilic gelling agent, a hydrophilic or lipophilic activator, a preservative, an antioxidant, a solvent, a fragrance, a filler, a blocking agent, a pigment, an odorant, and a dye, in addition to the at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts.

Another aspect of the present invention provides a food composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient. In the present invention, the composition may enhance skin elasticity and reduce wrinkles by promoting differentiation of human adipose-derived stem cells into adipose cells or proliferation of human adipose-derived stem cells.

The formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex, Aloe vera, extract, fraction, skin elasticity enhancement, and skin wrinkle reduction are as described above.

Also, the present invention provides a food composition for skin elasticity enhancement and skin wrinkle reduction including a sitologically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll as an active ingredient.

In the present invention, the “sitologically acceptable salt” is in the form of a salt in which formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll is combined with another substance, and refers to a substance capable of exhibiting sitologically similar activity to that of the compounds.

The food composition of the present invention may further include a sitologically acceptable supplementary food additive in addition to the at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts.

In the present invention, the “supplementary food additive” refers to a component supplementarily added to food and may be appropriately selected by those skilled in the art to prepare a health functional food in each formulation. Examples of the supplementary food additive may include various nutrients, vitamins, minerals (electrolytes), flavors such as synthetic and natural flavors, colorants and fillers, pectic acid and salts thereof, alginic acid and salts thereof, organic acids, protective colloidal thickeners, pH regulators, stabilizers, antiseptics, glycerin, alcohols, and carbonating agents used in soda, without being limited thereto.

A functional food composition of the present invention may include a health functional food. In the present invention, the “health functional food” refers to a food including raw materials or ingredients beneficial to the human body and prepared into formulations such as tablets, capsules, powders, granules, liquids, and pills. In this regard, “functional” means controlling of nutrients for structures and functions of the human body or obtaining beneficial effects in hygienic use such as in physiological functions. The health functional food of the present invention may be prepared by way of a method commonly used in the art, and raw materials and ingredients typically used in the art may be added thereto for the preparation of the health functional food. In addition, the health functional food may be prepared into any formulation that is regarded as a health functional food without limitation. The food composition of the present invention may be prepared into various formulations. Due to advantages over general drugs in that the food composition is free of side effects which might occur upon long-term intake of drugs, because it is manufactured using food ingredients and has high portability, the health functional food composition may be ingested as an aid for enhancing skin elasticity and reducing skin wrinkles.

The form of the health functional food according to the present invention is not limited, and the health functional food may include all food types commonly used in the art and may be used interchangeably with any term known in the art such as functional food. In addition, the health functional food of the present invention may be prepared by mixing any suitable auxiliary ingredients and known additives that may be contained in food and appropriately selected by one of ordinary skill in the art. Examples of the food to which the suitable auxiliary ingredients and the additives may be added may include meats, sausages, breads, chocolates, candies, snacks, cookies, pizzas, ramens, other noodles, gums, dairy products including ice cream, various kinds of soups, beverages, teas, drinks, alcoholic beverages, and vitamin complexes, and may also include foods used as animal feeds.

Another aspect of the present invention provides a pharmaceutical composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient. In the present invention, the composition may enhance skin elasticity and reduce skin wrinkles by promoting differentiation of human adipose-derived stem cells into adipose cells. The formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex, Aloe vera, extract, fraction, skin elasticity enhancement, and skin wrinkle reduction are as described above.

Also, the present invention provides a pharmaceutical composition for skin elasticity enhancement and skin wrinkle reduction including a pharmaceutically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, or xanthophyll as an active ingredient.

In the present invention, the “pharmaceutically acceptable salt” is in the form of a salt in which formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll is combined with another substance, and refers to a substance capable of exhibiting pharmaceutical activity similar to that of the compounds.

The pharmaceutical composition of the present invention may be used as a single formulation or combined with various drugs known to have effects on enhancing skin elasticity and reducing skin wrinkles as a composite formulation, and the composition may be formulated using a pharmaceutically acceptable carrier or excipient into a single-dose dosage form or a unit dosage form in a multidose container.

The pharmaceutical composition may be administered in a pharmaceutically effective amount. The “pharmaceutically effective amount” refers to an amount sufficient for treatment of diseases at a reasonable benefit/risk ratio applicable to a medical treatment, and the level of the effective dose may be determined based on factors including the kind of an individual, severity of illness, age or gender of the individual, drug activity, sensitivity to drug, administration time, administration route, excretion rate, length of treatment, and drug(s) concurrently used in combination, and other factors well known in the medical field.

Another aspect of the present invention provides an over-the-counter (OTC) composition for skin elasticity enhancement and skin wrinkle reduction including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient. In the present invention, the composition may enhance skin elasticity and reduce wrinkles by promoting proliferation of human adipose-derived stem cells into adipose cells. The formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion, rice bran, bamboo, carrot, Hibiscus, Magnoliae cortex, Aloe vera, extract, fraction, skin elasticity enhancement, and skin wrinkle reduction are as described above.

Also, the present invention provides an OTC composition for skin elasticity enhancement and skin wrinkle reduction including a pharmaceutically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll as an active ingredient. The “pharmaceutically acceptable salt” is as described above.

The OTC composition of the present invention may further include a pharmaceutically acceptable carrier, excipient, or diluent in addition to the above-described ingredients, if required. The pharmaceutically acceptable carrier, excipient, or diluent is not particularly limited as long as the effects of the present invention are not impaired thereby and may include, for example, a filler, an extender, a binder, a humectant, a disintegrant, a surfactant, a lubricant, a sweetener, a fragrance, or a preservative.

The “pharmaceutically acceptable carrier” may refer to a carrier, excipient, or diluent that does not impair biological activity or properties of the introduced compound while not irritating an organism, specifically a carrier which is not naturally occurring. The types of the carrier available in the present invention are not particularly limited, and any pharmaceutically acceptable carriers commonly used in the art may be used. Non-limiting examples of the carrier may include saline, sterilized water, Ringer's solution, buffered saline, albumin injection solution, dextrose solution, maltodextrin solution, glycerol, and ethanol, which may be used alone or in a combination of at least two thereof.

The composition including the pharmaceutically acceptable carrier may be prepared into various formulations suitable for oral or parenteral administration. For formulation, a typically used diluent or excipient such as a filler, an extender, a binder, a humectant, a disintegrant, or a surfactant may be used. Specifically, solid formulations for oral administration may include tablets, pills, powder, granules, capsules, and the like, and these solid formulations may be prepared by mixing the compound with at least one excipient, for example, starch, calcium carbonate, sucrose, lactose, or gelatin. In addition to simple excipients, a lubricant such as magnesium stearate or talc may also be used. Liquid formulations for oral administration may be suspensions, formulations for internal use, emulsions, syrups, or the like, and may include various excipients such as humectants, sweeteners, fragrances, and preservatives in addition to simple diluents commonly used in the art such as water or liquid paraffin. Formulations for parenteral administration may include sterile aqueous solutions, non-aqueous solvents, suspensions, emulsions, lyophilizates, suppositories, and the like. The non-liquid solutions and suspensions may be propylene glycol, polyethylene glycol, vegetable oils such as olive oil, injectable esters such as ethyl oleate, or the like. Bases for the suppositories may be Witepsol, Macrogol, Tween 61, cacao butter, laurin butter, glycerogelatin, or the like.

The OTC composition of the present invention may be a personal hygiene product, a shower foam, an ointment, a wet tissue, a coating agent, or the like, but is not limited thereto, and formulating methods, dosages, methods of use, components, and the like of the OTC composition may be appropriately selected from conventional techniques known in the art.

In addition, the OTC composition of the present invention may be used in a method of reducing skin wrinkles, the method including applying the composition to the skin of an individual. The individual may include mammals such as rats, livestock, and humans, without limitation.

Another aspect of the present invention provides a composition for promoting differentiation of human adipose-derived stem cells including at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, fractions of the Magnoliae cortex extract, and fractions of the Aloe vera extract as an active ingredient. In the present invention, the composition may enhance skin elasticity and reduce skin wrinkles by promoting differentiation of human adipose-derived stem cells into adipose cells. The formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion, rice bran, bamboo, carrot, Hibiscus, Magnoliae cortex, Aloe vera, extract, fraction, skin elasticity enhancement, and skin wrinkle reduction are as described above.

Another aspect of the present invention provides a method of enhancing skin elasticity, the method including administering the composition to an individual other than a human.

As used herein, the term “administration” refers to introduction of the composition of the present invention into a patient by way of any appropriate method. The composition may be administered via any known route as long as the composition is able to reach a target tissue. The composition may be administered via intraperitoneal administration, intravenous administration, intramuscular administration, subcutaneous administration, intradermal administration, oral administration, topical administration, or intranasal administration, without being limited thereto. Since the composition of the present invention has the effect on skin elasticity enhancement and skin wrinkle reduction, the composition may be administered by applying the composition to the skin.

The composition of the present invention may be administered daily or at an interval in a bolus or in multiple doses twice to three times per day. In addition, the composition of the present invention may be used alone or in combination with another drug treatment to enhance skin elasticity and reduce skin wrinkles. It is important to administer a minimum amount sufficient for obtaining the maximum effect without side effects in consideration of all of the above-described factors, and the amount may be easily determined by those skilled in the art.

As used herein, the term “individual” refers to any animal including humans, rats, mice, and livestock who have reduced skin elasticity and have or may have skin wrinkles. Specific examples thereof may be mammals including humans, but are not limited thereto.

### DETAILED DESCRIPTION OF THE INVENTION

Hereinafter, the present invention will be described in more detail with reference to the following examples. However, the following examples are merely presented to exemplify the present invention, and the scope of the present invention is not limited thereto.

### Example 1: Preparation of Extract of Magnoliae cortex and Aloe vera

Magnoliae cortex was purchased from Bolak Co., Ltd. (Hwaseong City, Gyeonggi Province, Republic of Korea). Magnoliae cortex powder was dissolved in a solvent such as DPG and DMSO and filtered using a 0.2 μm syringe filter, and the filtrate was used as a Magnoliae cortex extract.

For preparation of Aloe vera extract according to the present example, Aloe vera was purchased from a domestic farm and extracted once using 70% methanol at 70° C. at a ratio of 1:10.

### Example 2: Confirmation of Effect of Formononetin, Acetyl Mandelic Acid, and Magnoliae cortex Extract on Promoting Differentiation of Human Adipose-Derived Stem Cells

Human adipose-derived stem cells were purchased from Lonza, Inc. (Walkersvile, Md., USA) and cultured according to guidelines of Lonza, Inc. Differentiation into adipose cells was evaluated using a differentiation medium recommended by Lonza, Inc. As a positive control, the cells were treated with 1 μM rosiglitazone together with the differentiation medium. In order to induce differentiation of human adipose-derived stem cells into adipose cells, a cell culture solution was removed at 3 to 5 days after cell culturing was initiated, and the cells were treated with formononetin, acetyl mandelic acid, Magnoliae cortex extract, and the positive control for 14 days.

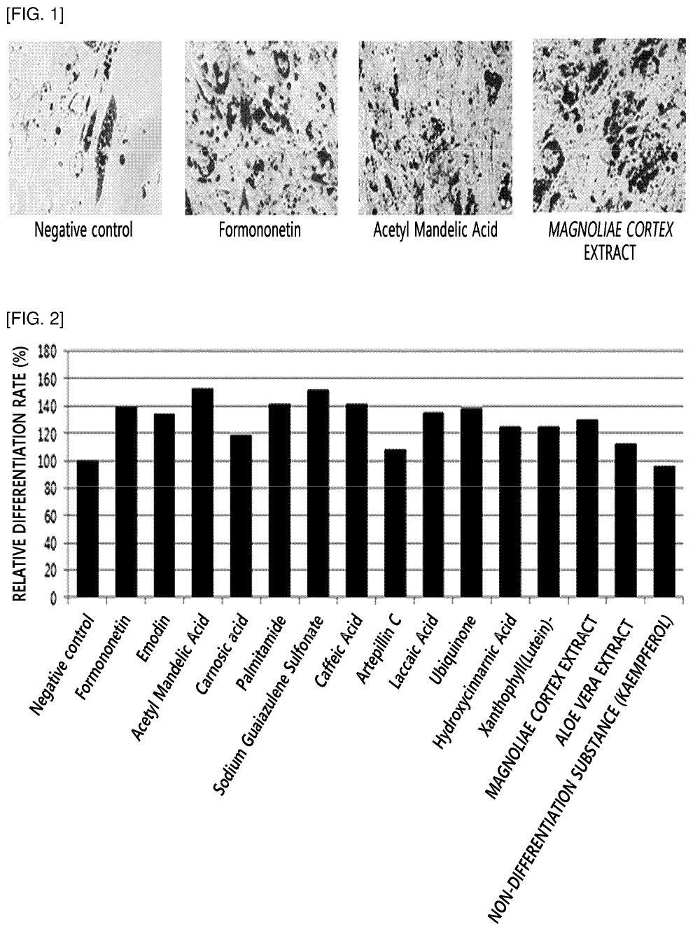

At 14 days after differentiation was initiated, the cell culture solution was removed, and the cells were washed with a phosphate buffer (DPBS) and immobilized using 10% formalin. The immobilized cells were observed using a microscope after staining fats and lipids contained in the adipose cells with Oil Red 0. As a result, it was confirmed that the effects of the representative substances of formononetin, acetyl mandelic acid, and Magnoliae cortex extract on differentiation of human adipose-derived stem cells into adipose cells were superior to those of the negative control treated only with the differentiation medium (FIG. 1).

### Example 3: Quantitative Confirmation of Effect of 12 Types of Compounds Including Formononetin and Magnoliae cortex Extract and Aloe vera Extract on Promoting Differentiation of Human Adipose-Derived Stem Cells

Cultured human adipose-derived stem cells were treated with 12 types of the compounds including formononetin and a Magnoliae cortex extract and an Aloe vera extract in the same manner as in Example 2. Stained Oil Red 0 was eluted using a 100% isopropanol solution, and absorbance thereof was measured at a wavelength of 490 nm and quantified. The results are shown in FIG. 2. Based thereon, all of the 14 types of the substances including formononetin according to the present invention exhibited significant increases in relative differentiation rates compared to the negative control treated only with the differentiation medium. It was confirmed that the substances according to the present invention promoted differentiation of human adipose-derived stem cells into adipose cells and effectively promoted differentiation into adipose cells compared to non-differentiation substances such as kaempferol. Thus, the compositions including the substances according to the present invention may be used for anti-aging purposes to enhance skin elasticity and reduce skin wrinkles by promoting differentiation of human adipose-derived stem cells and increasing the number of subcutaneous fat cells.

### Example 4: Confirmation of Effect of Magnoliae cortex Extract on Lip Wrinkle Reduction

In order to identify effects of Magnoliae cortex extract prepared in Example 1 on skin wrinkle reduction, lip winkles and under-eye wrinkles of subjects (total number of subjects: 12) were treated with the Magnoliae cortex extract.

The lip wrinkles were measured via Janus photography by designating the most protruding portion of a lower lip by a predetermined length and obtaining a standard deviation of a contrast value. The standard deviation of the contrast value was in the range of 3 to 12, and it was confirmed that more deep wrinkles indicate greater standard deviations. The under-eye wrinkles were measured via Janus photography using a wrinkle analysis program thereof, i.e., by calculating an area of wrinkles. It was confirmed that a wrinkle value was in the range of 7 to 44.

A change of 1 or more in the lip wrinkle values and a change of 4 or more in the under-eye wrinkle values were classified as improved result values. The criteria for classification were determined as ½ of a change of 2 for lip wrinkles and a change of 8 for under-eye wrinkles, which are values clearly distinguished by visual observation.

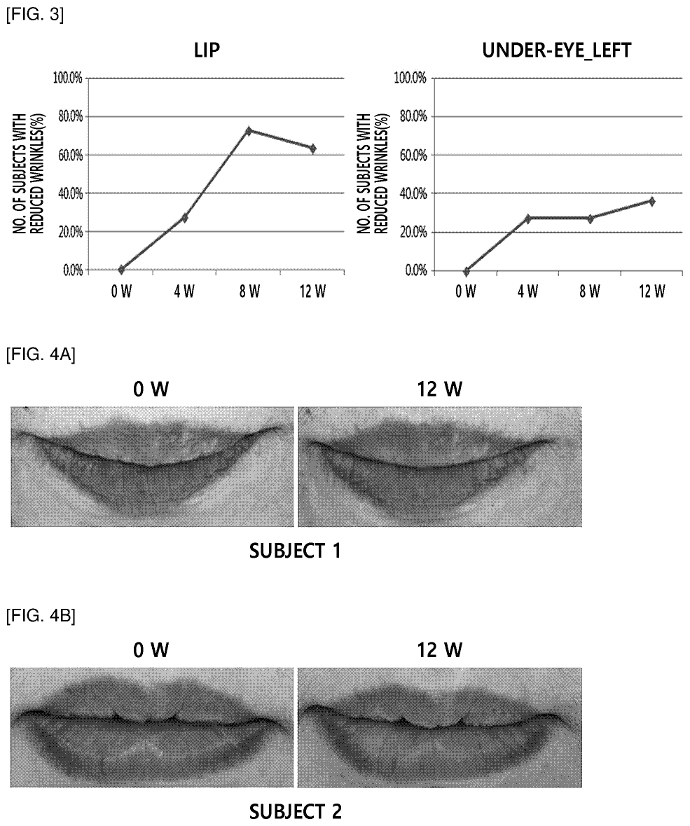

As a result of treating lip winkles with the Magnoliae cortex extract, wrinkle reduction was identified from the 4th week, a maximum number (8) of the subjects was identified at the 8th week, and a similar effect was maintained until the 12th week. As a result of treating under-eye wrinkles with the Magnoliae cortex extract, wrinkle reduction was observed at a similar rate to that of the lip wrinkles until the 4th week, and an increase in the number of the subjects with reduced wrinkles was confirmed until the 12th week, although an increasing rate was low (FIG. 3).

Also, it was confirmed that after treating the lip wrinkles of the subjects with the Magnoliae cortex extract, the effects on wrinkle reduction were maintained until the 12th week, and elasticity of the lip skin was enhanced (FIG. 4). This indicates that the Magnoliae cortex extract actually has effects on skin wrinkle reduction and skin elasticity enhancement by promoting differentiation of human adipose-derived stem cells and increasing the number of subcutaneous fat cells.

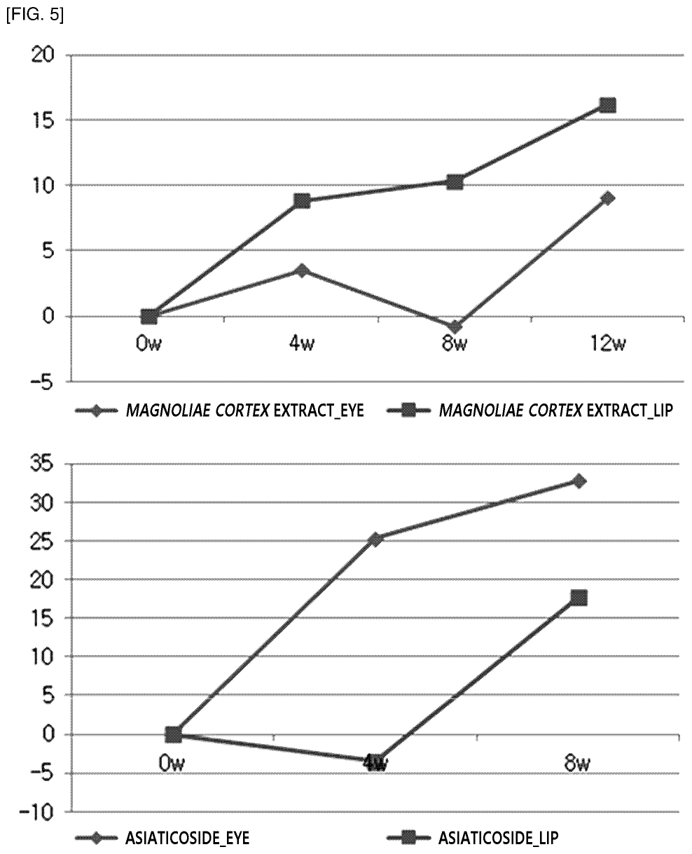

As a result of comparing effects of the Magnoliae cortex extract on reducing lip wrinkles and under-eye wrinkles with those of asiaticoside, which is a conventional wrinkle-reducing substance, it was confirmed that the Magnoliae cortex extract exhibited higher wrinkle reduction speed and wrinkle reduction rate for lip wrinkles than for under-eye wrinkles, while asiaticoside exhibited higher wrinkle reduction speed and wrinkle reduction rate for under-eye wrinkles than for lip wrinkles (FIG. 5).

Based thereon, it may be confirmed that the Magnoliae cortex extract had effects on skin wrinkle reduction, particularly on lip wrinkle reduction.

### Example 5: Preparation of Extracts of Onion, Rice Bran, Bamboo, Carrot, and Hibiscus

In the present invention, each of onion, rice bran, bamboo, carrot, and Hibiscus was subjected to extraction using methanol, and then dried to prepare extracts thereof in a powder form.

### Example 6: Confirmation of Effects of Compound and Extract on Promoting Proliferation of Human Adipose-Derived Stem Cells

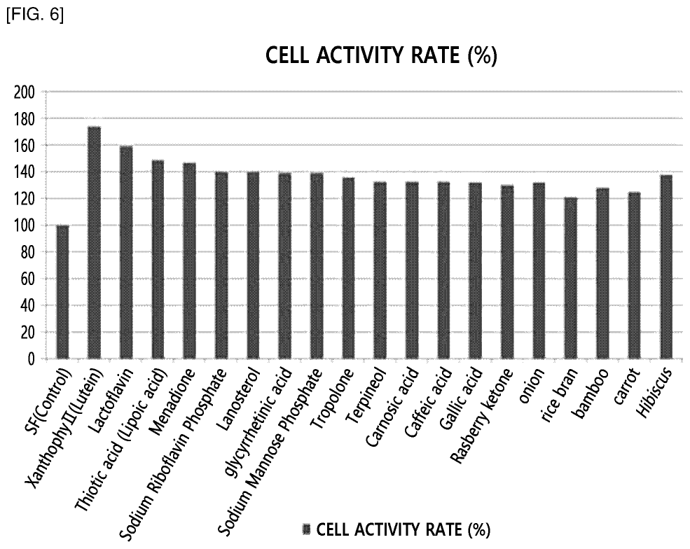

Human adipose-derived stem cells were purchased from Lonza, Inc. (Walkersville, Md., USA) and incubated in a CO₂ incubator at 37° C. under 5% CO₂ conditions. After the human adipose-derived stem cells were treated with 0.2 ppm, 2 ppm, and 20 ppm, respectively, cell activities thereof were identified using a CCK-8 kit (Dojindo). As a result, respective substances showed the highest activities at concentrations shown in Table 1 below and higher cell activity rates than those of all non-treated (control) groups by 20% or more (FIG. 6).

TABLE 1

| substance | concentration |
|---|---|
| Xanthophyll (Lutein) | 20 ppm |
| Lactoflavin | 20 ppm |
| Thioctic acid (Lipoic acid) | 20 ppm |
| Menadione | 0.2 ppm |
| Sodium Riboflavin Phosphate | 20 ppm |
| Lanosterol | 2 ppm |
| Glycyrrhetinic acid | 20 ppm |
| Sodium Mannose Phosphate | 2 ppm |
| Tropolone | 2 ppm |
| Terpineol | 20 ppm |
| Carnosic acid | 2 ppm |
| Caffeic acid | 20 ppm |
| Gallic acid | 20 ppm |
| Rasberry ketone | 20 ppm |
| onion | 2 ppm |
| rice bran | 20 ppm |
| bamboo | 2 ppm |
| carrot | 20 ppm |
| Hibiscus | 20 ppm |

Therefore, since a total of 19 compounds or extracts significantly promote proliferation of human adipose-derived stem cells, they may be used for anti-aging purposes by enhancing skin elasticity and reducing skin wrinkles.

The above description of the present invention is provided for the purpose of illustration, and it would be understood by those skilled in the art that various changes and modifications may be made without changing the technical conception and essential features of the present invention. Thus, it is clear that the above-described embodiments are illustrative in all aspects and do not limit the present invention. The various embodiments disclosed herein are not intended to be limiting, with the true scope and spirit being indicated by the following claims. The present invention is to be limited only by the terms of the appended claims, along with the full scope of equivalents to which such claims are entitled.

1. A cosmetic composition for skin elasticity enhancement and skin wrinkle reduction comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

2. The cosmetic composition of claim 1, wherein the composition comprises a cosmetically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll.

3. The cosmetic composition of claim 1, wherein an amount of the substance is in the range of 0.0001 wt % to 10 wt % (w/w) based on a total weight of the composition.

4. The cosmetic composition of claim 1, wherein the extract is obtained using water, a C₁-C₆ alcohol, or a mixed solvent thereof.

5. The cosmetic composition of claim 1, wherein the substance promotes differentiation of human adipose-derived stem cells into adipose cells.

6. The cosmetic composition of claim 5, wherein the substance comprises at least one selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, Aloe vera extract, fractions of the Magnoliae cortex extract, and fractions of the Aloe vera extract.

7. The cosmetic composition of claim 1, wherein the substance promotes proliferation of human adipose-derived stem cells.

8. The cosmetic composition of claim 7, wherein the substance comprises at least one selected from the group consisting of raspberry ketone, gallic acid, caffeic acid, carnosic acid, terpineol, sodium mannose phosphate, tropolone, glycyrrhetinic acid, lanosterol, sodium riboflavin phosphate, menadione, thioctic acid, lactoflavin, xanthophyll, onion extract, rice bran extract, bamboo extract, carrot extract, Hibiscus extract, and fractions of the extracts.

9. The cosmetic composition of claim 1, wherein the composition is prepared in a formulation selected from a softening lotion (nourishing emulsion), astringent lotion, nourishing lotion, nourishing cream, massage cream, essence, pack, transdermal patch, transdermal gel, powder, ointment, suspension, emulsion, spray, or serum.

10. A food composition for skin elasticity enhancement and skin wrinkle reduction comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

11. The food composition of claim 10, wherein the composition comprises a sitologically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll.

12. A pharmaceutical composition for skin elasticity enhancement and skin wrinkle reduction comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

13. The pharmaceutical composition of claim 12, wherein the composition comprises a pharmaceutically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll as an active ingredient.

14. An over-the-counter composition for skin elasticity enhancement and skin wrinkle reduction comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, raspberry ketone, gallic acid, terpineol, sodium mannose phosphate, tropolone, glycyrrhetinic acid, lanosterol, sodium riboflavin phosphate, menadione, thioctic acid, lactoflavin, onion extract, rice bran extract, bamboo extract, carrot extract, Hibiscus extract, Magnoliae cortex extract, Aloe vera extract, and fractions of the extracts as an active ingredient.

15. The over-the-counter composition of claim 14, wherein the composition comprises a pharmaceutically acceptable salt of formononetin, emodin, artepillin C, carnosic acid, palmitamide, sodium guaiazulene sulfonate, caffeic acid, acetyl mandelic acid, laccaic acid, ubiquinone, hydroxycinnamic acid, raspberry ketone, gallic acid, terpineol, sodium mannose phosphate, tropolone, glycyrrhetinic acid, lanosterol, sodium riboflavin phosphate, menadione, thioctic acid, lactoflavin, or xanthophyll as an active ingredient.

16. A composition for promoting differentiation of human adipose-derived stem cells comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, Aloe vera extract, a fraction of the Magnoliae cortex extract, and a fraction of the Aloe vera extract as an active ingredient.

17. A composition for promoting proliferation of human adipose-derived stem cells comprising at least one substance selected from the group consisting of raspberry ketone, gallic acid, caffeic acid, carnosic acid, terpineol, sodium mannose phosphate, tropolone, glycyrrhetinic acid, lanosterol, sodium riboflavin phosphate, menadione, thioctic acid, lactoflavin, xanthophyll, onion extract, rice bran extract, bamboo extract, carrot extract, Hibiscus extract, and fractions of the extracts as an active ingredient.

18. (canceled)

19. A cosmetic composition for elasticity enhancement and wrinkle reduction of lip skin comprising at least one substance selected from the group consisting of formononetin, emodin, artepillin C, carnosic acid, palmitamide, sodium guaiazulene sulfonate, caffeic acid, acetyl mandelic acid, laccaic acid, ubiquinone, hydroxycinnamic acid, xanthophyll, Magnoliae cortex extract, Aloe vera extract, a fraction of the Magnoliae cortex extract, and a fraction of the Aloe vera extract as an active ingredient.

20. The cosmetic composition of claim 19, wherein the active ingredient of the cosmetic composition for elasticity enhancement and wrinkle reduction of lip skin comprises at least one substance selected from the group consisting of the Magnoliae cortex extract and the fraction of the Magnoliae cortex extract.

21. The cosmetic composition of claim 19, wherein the composition is prepared in a formulation selected from a lipstick, a lip gloss, a lip tint, or a lip balm.
